(19) United States
(12) Patent Application Publication
(10) Pub. No.: US 2026/0289952 A1
(43) Pub. Date: Sep. 24, 2026

(54) CUSTOMIZING A MACHINE LEARNING MODEL TO PROCESS A SELECTED SUBSET OF FEATURES TO PREDICT GEOGRAPHIC ATROPHY PROGRESSION

(71) Applicant: Genentech, Inc., South San Francisco, CA (US)

(72) Inventors: Julia Gabriella CLUCERU, San Francisco, CA (US); Simon Shang GAO, San Francisco, CA (US); Neha Sutheekshna ANEGONDI, Fremont, CA (US)

(21) Appl. No.: 19/685,269

(22) Filed: May 22, 2026

Related U.S. Application Data
(63) Continuation of application No. PCT/US24/59249, filed on Dec. 9, 2024.
(60) Provisional application No. 63/607,547, filed on Dec. 7, 2023.
(60) Provisional application No. 63/642,118, filed on May 3, 2024.

(51) Int. Cl. G06V 10/46; G06T 7/00; G06V 10/20; G06V 10/54; G06V 10/766; G06V 10/776

(52) U.S. Cl. CPC G06V 10/467; G06T 7/0012; G06V 10/255; G06V 10/54; G06V 10/766; G06V 10/776; G06T 2207/10064; G06T 2207/30041

(57) ABSTRACT

Methods and systems for evaluating geographic atrophy using a customized machine learning model. A set of fundus autofluorescence (FAF) images for a subject are received. Input for the machine learning model is generated using the plurality of FAF images. The input includes a subset of features that have been selected from a plurality of features, which include a plurality of image features. The plurality of image features includes at least one of a plurality of shape features and a plurality of texture features. Selection of the subset of features from the plurality of features is based on evaluating performance of a set of trained models that are used to process at least two different combinations of the plurality of features. A set of geographic atrophy (GA) progression parameters based on the input are predicted via the customized machine learning model.

### CROSS-REFERENCE TO RELATED APPLICATIONS

This application is a continuation of International Application No. PCT/US 2024/059249, filed Dec. 9, 2024, which claims priority to U.S. Provisional Application. No. 63/607,547, filed Dec. 7, 2023 and U.S. Provisional Application No. 63/642,118, filed May 3, 2024, each of which is incorporated herein by reference in its entirety. Further, this application is related to U.S. patent application Ser. No. 18/513,106, filed Nov. 17, 2023, which is a continuation of International Application No. PCT/US 2022/029699, filed May 17, 2022, which claims priority to U.S. Provisional Patent Application No. 63/189,679, filed May 17, 2021, and also related to U.S. Provisional Patent Application No. 63/601,949, filed Nov. 22, 2023, each of which is incorporated herein by reference in its entirety.

### FIELD

This description is generally directed toward the prediction of geographic atrophy progression. More specifically, this description provides methods and systems for building machine learning model to predict geographic atrophy progression with improved performance using a selected set of features.

### BACKGROUND

Age-related macular degeneration (AMD) is a leading cause of vision loss in patients 50 years or older. Geographic atrophy (GA) is one of two advanced stages of AMD and is characterized by progressive and irreversible loss of choriocapillaris, retinal pigment epithelium (RPE), and photoreceptors. GA lesions can be detected using several different imaging modalities, including color fundus photography, fluorescein angiography, fundus autofluorescence (FAF), near-infrared reflectance, and optical coherence tomography (OCT). FAF is an imaging modality that allows topographic mapping of lipofuscin distribution in the retinal pigment epithelium cell monolayer and other fluorophores that may occur with disease in the outer retina and the subneurosensory space. GA progression varies between patients, and currently, no widely accepted treatment for preventing or slowing down the progression of GA exists. Therefore, evaluating GA progression in individual patients may be important to researching GA and developing an effective treatment.

### SUMMARY

In one or more embodiments, a method for evaluating geographic atrophy using a customized machine learning model is provided. A set of fundus autofluorescence (FAF) images for a subject are received. Input for the machine learning model is generated using the plurality of FAF images. The input includes a subset of features that have been selected from a plurality of features. The plurality of features includes a plurality of image features. The plurality of image features includes at least one of a plurality of shape features and a plurality of texture features. The subset of features includes fewer features than the plurality of features. Selection of the subset of features from the plurality of features is based on evaluating performance of a set of trained models that are used to process at least two different combinations of the plurality of features. A set of geographic atrophy (GA) progression parameters based on the input are predicted via the customized machine learning model.

In one or more embodiments, a method for generating a customized machine learning model is provided. A plurality of fundus autofluorescence (FAF) images for a plurality of subjects is received. A plurality of mask images based on the plurality of FAF images is generated. For each FAF image of the plurality of FAF images, a plurality of image features based on the plurality of mask images is generated. The plurality of image features includes a plurality of shape features and a plurality of texture features. A set of initial machine learning models is trained to predict growth rate of a geographic atrophy (GA) lesion using a plurality of features. The plurality of features includes the plurality of image features and a plurality of clinical features. A plurality of scores for the plurality of features is generated, in which the plurality of scores indicates a ranking of importance of the plurality of features to the predicted growth rate. A customized machine learning model is built that predicts growth rate using a subset of features selected from the plurality of features based on the plurality of scores.

In one or more embodiments, system for generating a customized machine learning model is provided. The system comprises at least one data processor; and at least one memory storing instructions, which when executed by the at least one data processor, cause the processor to: receive a plurality of fundus autofluorescence (FAF) images for a plurality of subjects; generate a plurality of mask images based on the plurality of FAF images; generate, for each FAF image of the plurality of FAF images, a plurality of image features based on the plurality of mask images, wherein the plurality of image features includes a plurality of shape features and a plurality of texture features; train a set of initial machine learning models to predict growth rate of a geographic atrophy (GA) lesion using a plurality of features, wherein the plurality of features includes the plurality of image features and a plurality of clinical features; generate a plurality of scores for the plurality of features in which the plurality of scores indicates a ranking of importance of the plurality of features to the predicted growth rate; and build a customized machine learning model that predicts growth rate using a subset of features selected from the plurality of features based on the plurality of scores.

In one or more embodiments, a system comprises at least one data processor; and at least one memory storing instructions, which when executed by the at least one data processor, result in operations comprising any one or more of the methods described herein or a portion thereof.

In one or more embodiments, a non-transitory computer readable medium storing instructions is provided, which when executed by at least one data processor, result in comprising any one or more of the methods described herein or a portion thereof.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a prediction system 100 in accordance with various embodiments.

FIG. 2 is a block diagram of the feature extraction module and the plurality of image features from FIG. 1 in accordance with one or more embodiments.

FIG. 3 is a flowchart of a process for evaluating geographic atrophy using a customized machine learning model in accordance with one or more example embodiments.

FIG. 4 is a flowchart of a process for generating a customized machine learning model in accordance with one or more example embodiments.

FIG. 5 is an example illustration of a lesion mask image in accordance with one or more embodiments.

FIG. 6 is a block diagram of a computer system in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The ability to accurately predict geographic atrophy (GA) progression based on baseline assessments may be useful in many different scenarios. Parameters associated with GA progression include lesion growth rate, baseline lesion area, or future lesion area (e.g., at a future point in time). Baseline lesion area is the total area of a GA lesion (e.g., in mm²). Baseline lesion area has been shown to be an indicator of GA progression, which may be evaluated based on lesion growth rate. Lesion growth rate, which may be also referred to herein as GA lesion growth rate or growth rate, is the change in lesion area over some time period. Oftentimes, the growth rate is annualized (e.g., mm₂/year).

Being able to automatically predict lesion growth rate and optionally, baseline and/or future lesion area, an input retinal imaging and a machine learning (ML) model may help improve patient screening, enrichment, and/or stratification in clinical trials where the goal is to slow GA progression, thereby allowing for improved assessment of treatment effects. Improving predictions of such GA progression parameters (e.g., lesion growth rate, baseline lesion area, future lesion area) may also improve clinical trial efficacy through, for example, without limitation, allowing for covariate adjustment during analysis. Covariate adjustment may be used to reduce the variance of the treatment effect estimate in the clinical trial and increase the power of the clinical trial. Additionally, in some cases, predictions of GA progression parameters may be used to understand disease pathogenesis via correlation to genotypic or phenotypic signatures.

A GA lesion can be imaged by various imaging modalities including, but not limited to, fundus autofluorescence (FAF). For example, fundus autofluorescence (FAF) images may be input into one or more ML models to predict baseline lesion area, lesion growth rate, future lesion area, or a combination thereof. The FAF images may be baseline FAF images that are taken at a baseline point in time. The baseline point in time may be the beginning of the clinical trial, the time of the initial assessment, a time just prior to a first administration of treatment, a time coincident with the first administration of treatment, a same day as the first administration of treatment, or some other baseline point in time. While machine learning may be used to analyze and predict GA progression based on FAF images, in certain cases, existing machine learning methods may be unable to provide the desired level of accuracy without consuming more computing resources or taking longer than desired.

Thus, the embodiments described herein recognize that it may be desirable to build a customized machine learning model that is capable of having the desired level of performance while also reducing the overall computing resources (e.g., processing power, memory, etc.) that may need to be consumed and/or reducing the overall time needed for predicting GA progression based on input FAF imaging. Accordingly, the embodiments recognize and take into account that it may be desirable to have methods and systems for understand how ML models use one or more baseline FAF images to predict GA progression and the different types of features that may be important to making accurate GA progression predictions. For example, it may be desirable to understand which regions or features of a baseline FAF image contribute to the lesion growth rate that is predicted. Identifying which image regions or features that are relevant to (or drive) the prediction of lesion growth rate may help identify or localize new biomarkers, gain insight into GA pathology, develop trust that the ML model is not focusing on spurious or irrelevant image regions or features, and/or improve the performance of the ML model. Additionally, the identified image regions or features of images may be used to generate ML models that may more accurately predict GA growth rate.

The embodiments described herein provide methods and systems for evaluating the image regions or features of images (e.g., FAF images) that contribute to the prediction of one or more GA progression parameters (e.g., lesion growth rate, baseline lesion area, etc.) by a machine learning model (e.g., a linear regression model, XGBoost).

In one or more embodiments, an FAF image processor, which includes a ML model, is used to predict GA progression and feature contribution after having been used over multiple cycles to predict GA progression (e.g., growth rate) for different combinations of features that are input into the ML model. For example, the ML model may generate a feature contribution output (e.g., feature scores) that identifies which of the features used by the ML model for prediction contributed most to the prediction. This feature contribution output may then be used to identify modifications that can be made to the ML model to build a new, customized ML model with improved performance relying on a smaller subset of features. For example, the feature contribution output may be used to select a subset of features of the total number of features to be used as input by the ML model to predict GA progression. In this manner, the feature contribution output is used to narrow the focus of the new, customized ML model to reduce the time and computing resource expenditure of the new ML model. For example, the new, customized model ML may use fewer computing resources (e.g., processing power, memory, etc.) to process a smaller subset of features than would be needed to process a larger subset of features using the initial ML model, while maintaining a desired level of prediction accuracy.

### II. Example Systems for Geographic Atrophy (GA) Progression Prediction

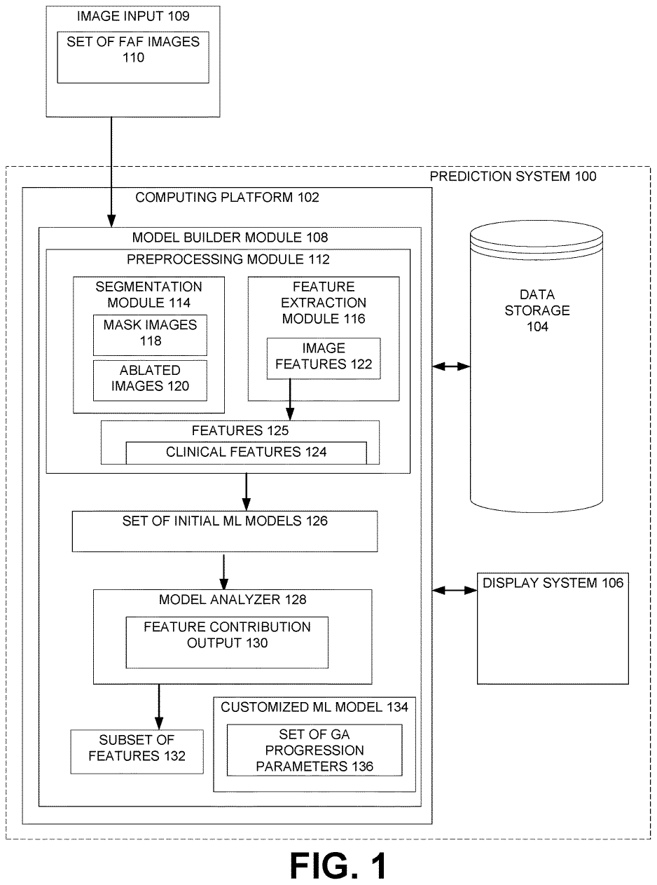

FIG. 1 is a block diagram of a prediction system 100 in accordance with various embodiments. Prediction system 100 is used to evaluate geographic atrophy (GA) lesions in the retinas of subjects. Prediction system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Prediction system 100 includes model builder module 108, which may be implemented using hardware, software, firmware, or a combination thereof. In one or more embodiments, model builder module 108 is implemented in computing platform 102. Model builder module 108 may also be referred to as model builder.

Model builder module 108 is used to build a customized model that receives as input, a selected subset of features that have been determined to be most important or that contribute the most to the prediction of GA progression (e.g., lesion growth rate).

Model builder module 108 receives image input 109 for processing. Image input 109 includes a plurality of retinal images. In one or more embodiments, image input 109 includes a plurality of retinal images that are generated at a baseline or reference point in time. In some embodiments, image input 109 may be referred to as baseline image input. In one or more embodiments, image input 109 includes plurality of fundus autofluorescence (FAF) images 110. The plurality of FAF images 110 may be, for example, a plurality of baseline FAF images.

A baseline image (e.g., baseline FAF) is an image captured at a baseline point in time. The baseline point in time may be the beginning of the clinical trial, the time of an initial assessment or initial clinic or clinical trial visit, a time just prior to a first administration of treatment, a time coincident with the first administration of treatment, a same day as the first administration of treatment, or some other baseline point in time.

Model builder module 108 processes the plurality of FAF images 110 using preprocessing module 112. Preprocessing module 112 may include segmentation module 114 and feature extraction module 116. Segmentation module 114 processes the plurality of FAF images 110 to generate plurality of mask images 118 where the plurality of mask images 118 includes, for each FAF image of the plurality of FAF images 110, a set of mask images. In some cases, a mask image may also be referred to as a segmented image. For example, the mask images 118 may include a lesion mask image that segments out the one or more GA lesions identifiable in the corresponding FAF image. For example, with a lesion mask image, the background may be black while the pixels identified as representing GA lesion may be made white. A rim mask image segments out the portion of the corresponding FAF image that represents a rim around the GA lesion(s). A background mask image segments out the portion of the corresponding FAF image that represents the background (not lesion or rim). In some cases, the FAF images 110 include a lesion and rim mask image that segments out both the lesion and rim.

In one or more embodiments, segmentation module 114 may further generate a plurality of ablated images 120. Ablated images 120 may include, for example, for each corresponding FAF image, an ablated image that blacks out the pixels that are not of interest. For example, for a lesion ablated image, all pixels from the FAF image except those representing the GA lesion may be blacked out (or ablated). For a rim ablated image, all pixels from the FAF image except those representing the rim of the GA lesion may be blacked out (or ablated). For a background ablated image, all pixels from the FAF image except those representing the background (and therefore not the lesion or rim) may be blacked out (or ablated).

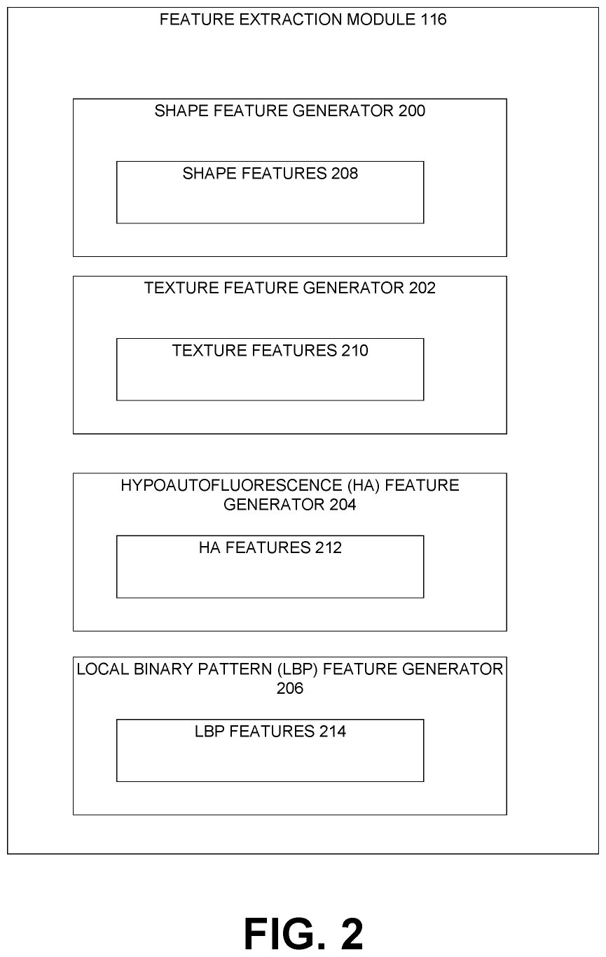

Feature extraction module 116 may receive as input the mask images 118, the ablated images 120, or both for processing to generate a plurality of image features 122. The plurality of image features 122 that can be derived from mask images 118 are described in greater detail in FIG. 2.

Preprocessing module 112 may also receive and process certain clinical information to form a plurality of clinical features 124. Clinical features 124 may include, for example, without limitation, age, sex, baseline lesion area as measured, an identification of whether the lesion is subfoveal or non-subfoveal, an indication of contiguity (e.g., unifocal or multifocal), baseline visual acuity (e.g., baseline best corrected visual acuity (BCVA)), baseline low-luminance deficit, baseline distance to fovea (e.g., distance to central fovea), smoking status, baseline peripheral reticular pigmentary degeneration (PRPD), one or more other clinical features, or a combination hereof.

Preprocessing module 112 may be used to form different combinations of inputs using plurality of image features 122 and plurality of clinical features 124. Together, plurality of image features 122 and plurality of clinical features 124 may be referred to as plurality of features 125. These combinations of inputs may be used to train a set of initial machine learning (ML) models 126 to predict GA progression (e.g., lesion growth rate). For example, each model in set of initial models 126 may be used to generate a predicted lesion growth rate for a given input formed from one or more image features of the plurality of image features 122, one or more clinical features of the plurality of clinical features 124, or a combination thereof. This “run” may be repeated for different combinations of features.

In one or more embodiments, model builder module 108 includes model analyzer 128 that is used to analyze the performance of and outputs of set of initial ML models 126 for the various feature combinations that are input into set of initial ML models 126. For example, set of initial ML models 126 may include a linear regression model, a linear regression model with Lasso, a linear regression model with Ridge, a linear regression model with Elastic Net; and XGBoost. In some cases, these models may output a ranking of importance for the features input into the model. Model analyzer 128 may be used to process these different rankings to generate feature contribution output 130 for plurality of features 125. Feature contribution output 130 may include, for example, a overall feature score for each of the plurality of features 125. In some cases, feature contribution output 130 may include these feature scores for plurality of features 125 for each different model in set of initial ML models 126. In other examples, feature contribution output 130 may include overall feature scores applicable across set of initial ML models 126.

In one or more embodiments, feature contribution output 130 may indicate which inputs were most important and which were the least important to prediction. For example, feature contribution output 130 may identify that certain ones of the plurality of features 125 were most important to the prediction of GA progression, providing insight into potential biomarkers for GA progression. In this manner, feature contribution output 130 may, for example, identify information that provides insight into GA pathology. Further, feature contribution output 130 may, for example, indicate whether a model of set of initial ML models 126 can be validated.

Thus, model analyzer 128 may evaluate model performance with respect to accuracy, precision, reliability, a coefficient of determination (r²), one or more other metrics, or a combination thereof. Evaluating the performance of these different models may help identify or localize biomarkers, gain insight into GA pathology, develop trust that a given ML model is not focusing on spurious or irrelevant image regions or features, and/or improve the performance of set of initial models 126. In some embodiments, model analyzer 128 computes the square of Pearson's correlation coefficient (r²) to evaluate the performance of set of initial ML models 126.

Model builder module 108 may use feature contribution output 130 to select subset of features 132 for use in building customized machine learning (ML) model 134. Subset of features 132 may contain fewer features than plurality of features 125. For example, subset of features 132 may include three, four, five, six, seven, eight, nine, ten or some other number of features. Customized ML model 134 may be one that is capable of predicting GA progression (e.g., via predicting lesion growth rate) for a given FAF image with a desired level of accuracy while also reducing the overall computing resources (e.g., processing power, memory, etc.) and time needed to go from FAF images to predicted GA progression. For example, by using only subset of features 132, the computing resources consumed by customized ML model 134 to predict lesion growth rate for a particular FAF image may be reduced as well as the computing resources consumed by feature extraction module 116 for the generation of subset of features 132 based on the given image.

Customized ML model 134 may be used to predict set of GA progression parameters 136. A GA progression parameter is one that is associated with (indicates or can be used to indicate) GA progression of a GA lesion. The GA lesion may be a continuous or discontinuous lesion. Set of GA progression parameters 136 may include one or more lesion area parameters, growth rate, or both. A lesion area parameter may be a baseline lesion area (e.g., a lesion area at the baseline point in time) or a future lesion area (e.g., lesion area at a future point in time) for the GA lesion. Growth rate (or lesion growth rate) may be the change in lesion area over a defined period of time. In some cases, growth rate may be annualized (e.g., mm₂/year).

FIG. 2 is a block diagram of feature extraction module 116 and a plurality of image features 122 from FIG. 1 in accordance with one or more embodiments. As discussed above, feature extraction module 116 may use mask images 118, ablated images 120, or both to generate image features 122.

Feature extraction module 116 may include, for example, without limitation, shape feature generator 200, texture feature generator 202, hypoautofluorescence (HA) feature generator 204, local binary pattern (LBP) feature generator 206, or a combination thereof. Each of these generators uses the mask images 118, the ablated images 120, the original FAF images 110, or a combination thereof.

Shape feature generator 200 is used to generate a plurality of shape features 208. Texture feature generator 202 is used to generate a plurality of texture features 210. HA feature generator 204 is used to generate a plurality of hypoautofluorescence (HA) features 212. The LBP feature generator 206 is used to generate a plurality of local binary pattern (LBP) features 214. Shape features 208, texture features 210, HA features 212, and LBP features 214 are examples of features in plurality of image features 122 from FIG. 1. In some cases, plurality of image features 122 may also include other types of features. For example, image features 122 may include a mean intensity feature that corresponds to the mean intensity of the pixels of the lesion or rim.

Shape features 208 may include, for example, without limitation, a measured GA lesion area, an identified number of lesions, a perimeter, a Feret max, a Feret min, and circularity. Shape feature generator 200 may analyze mask images 118 in order to generate shape features 208. For example, mask images 118 may include a lesion mask image for each corresponding FAF image of plurality of FAF images 110. Shape feature generator may use the lesion mask image to compute or otherwise quantify shape features 208. In one or more embodiments, convex hulls are used to further process mask images 118 by, for example, applying a convex hull around the segmented lesion of lesion mask image. The convex hull mask image uses a convex hull to capture the overall shape of the lesion. For multifocal lesions, circularity and Feret diameters (min and max) may be quantified using the average of all individual lesions weighted by an area (weighted average).

Texture features 210 may include, for example, without limitation, radiomics features that may be computed based on the mask images 118. Radiomics features may include, for example, at least one of a set of gray-level co-occurrence matrix features, a set of gray level size zone matrix features, a set of gray level run length matrix features, a set of neighboring gray tone difference matrix features, or a set of gray level dependence matrix features,

The HA features 212 may include various features that are generated by analyzing the FAF images 110. For example, the FAF images 110 may be processed using contrast-limited adaptive histogram equalization (CLAHE) processing. This processing may be a form a histogram normalization to ensure that a histogram-based intensity threshold from which to derive the HA regions of the FAF images. The histograms are quantified for both the rim region (e.g., as determined by the ablated images 120) of the FAF images and the background regions (e.g., as determined by the ablated images 120) of the FAF images. Two intensity thresholds are chosen to create a mask of HA regions. For these two masks, the number of HA regions and the total area of the HA regions are quantified, resulting in eight total HA features. In one or more embodiments, the thresholds may be selected between for example, intensity levels between about 120 and about 160. In some embodiments, these HA in the FAF images may be referred to as “speckle.”

Local binary pattern (LBP) evaluates whether points surrounding a central point are greater than or less than the center and gives a binary result. Edges can be found toward the center of an LBP histogram; corners can be found on either side of these edges; flat or homogenous sections can be found towards the edges of the LBP histogram.

The LBP features 214 may be quantified in the lesion and rim regions (e.g., as determined by the ablated images 120) of the FAF images 110. In one or more embodiments, two types of local binary patterns are computed on the lesion mask images and rim mask images of the mask images 118. These two types may be a uniform LBP and a rotation-invariant LBP. Histograms of the resulting LBP images may be computed with density normalization. Various features may be extracted from these LBP histograms, including standard deviation, skewness, kurtosis, inter quartile range (IQR), range, and intensity. Further, these features may be also computed for specific regions of the LBP histograms that are thought to correspond to the number of edges, corners, and flat regions of an FAF image. The LBP features computed for the overall LBP images as well as the specific LPB regions (e.g., edges, corners, flat regions) may form the full set of LPB features 214 in one or more embodiments. In some cases, the LPB features 214 include 92 features.

In one or more embodiments, the features in subset of features 132 from FIG. 1 that are selected from the various plurality of image features 122 shown in FIG. 2 may be selected based on importance. As previously discussed, the subset of features 132 may include three, four, five, six, seven, eight, nine, ten, or some other number of features. In one or more embodiments, subset of features 132 may include an LBP feature corresponding to a “range” computed for a corner region; an LPB feature corresponding to an inner quartile range (IQR) for a corner region; a low-luminance deficit feature (which is a clinical feature), an intensity feature for the lesion corresponding to the 10^{th} intensity percentile (e.g., the intensity value for the intensity of the pixels of the lesion at the 10^{th} percentile), an LPB feature corresponding to a minimum value for a flat region; a distance to central foveal feature (which is a clinical feature), a texture feature in the form of lesion short run high gray level emphasis, a rim Feret max feature (which is a shape feature), a lesion Feret min (which is a shape feature), and an LPB feature corresponding to a “range” computed for an edge region. In other embodiments, some portion of these ten identified features may be used. For example, the first three, four, five, six, seven, eight, or nine of these ten features as listed in the above listing may be used as inputs for the customized ML model 134 in FIG. 1.

### III. Example Methods for Geographic Atrophy (GA) Progression Prediction

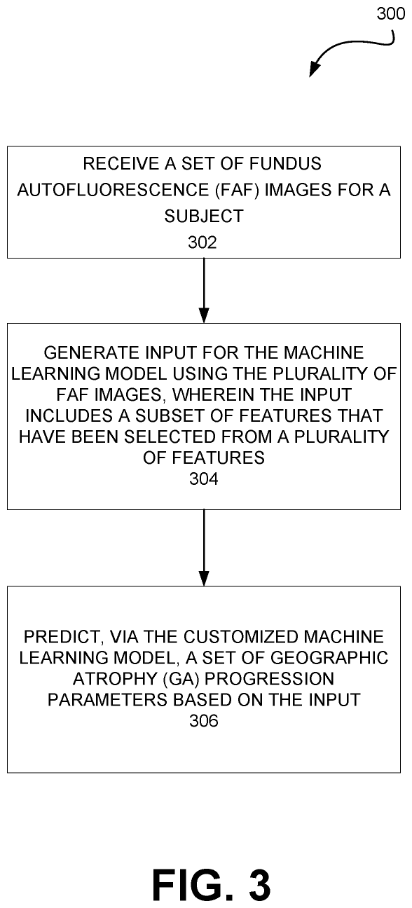

FIG. 3 is a flowchart of a process for evaluating geographic atrophy using a customized machine learning model in accordance with one or more example embodiments. Process 300 in FIG. 3 may be implemented using prediction system 100 described with respect to FIGS. 1 and 2. In one or more embodiments, at least some of the steps of the process 300 may be performed by the processors of a computer or a server implemented as part of prediction system 100. It is understood that additional steps may be performed before, during, or after the steps of process 300 discussed below. In addition, in some embodiments, one or more of the steps may also be omitted or performed in different orders.

Step 302 of process 300 includes receiving a set of fundus autofluorescence (FAF) images for a subject.

Step 304 of process 300 includes generating input for the machine learning model using the plurality of FAF images, wherein the input includes a subset of features that have been selected from a plurality of features. The input may include a subset of features that have been selected from a plurality of features. The plurality of features may include a plurality of image features and a plurality of clinical features. The plurality of image features may include at least one of a plurality of shape features and a plurality of texture features.

In some embodiments, the plurality of shape features may include at least two or more of a measured GA lesion area, an identified number of lesions, a perimeter, a Feret max, a Feret min, and circularity. In one or more embodiments, the plurality of texture features may include radiomics features that include at least one of a set of gray-level co-occurrence matrix features, a set of gray level size zone matrix features, a set of gray level run length matrix features, a set of neighboring gray tone difference matrix features, or a set of gray level dependence matrix features. The plurality of features may further include hypoautofluorescence (HA) features and local binary pattern features, as described in FIG. 2.

In one or more embodiments, generating the input may include generating a set of mask images based on the set of FAF images, wherein the set of mask images includes at least one of a lesion mask image, a rim mask image, a lesion and rim mask image, or a background mask image, and generating at least one feature of the subset of features using the set of mask images. In some embodiments, generating the input may further include generating at least one shape feature that is included in the subset of features using the set of mask images, generating at least one hyperautofluorescence (HA) feature that is included in the subset of features using the set of mask images, and/or generating a set of radiomics features that is included in the subset of features using the set of mask images.

In some embodiments, generating the input may further include computing a first local binary pattern for the lesion mask image and a second local binary pattern for the rim mask image, and generating a plurality of local binary pattern features that are included in the subset of features using the first local binary pattern and the second local binary pattern. For example without limitation, the first local binary pattern may be a uniform local binary pattern and the second local binary pattern may be a rotation-invariant local binary pattern. Various local binary pattern features may be computed based on these pattern images.

The subset of features may include fewer features than the plurality of features. Selection of the subset of features from the plurality of features may be based on evaluating performance of a set of trained models that are used to process at least two different combinations of the plurality of features.

The subset of features may include three, four, five, six, seven, eight, nine, ten, or some other number of features. In one or more embodiments, the subset of features may include an LBP feature corresponding to a “range” computed for a corner region; an LPB feature corresponding to an inner quartile range (IQR) for a corner region; a low-luminance deficit feature (which is a clinical feature), an intensity feature for the lesion corresponding to the 10^{th} intensity percentile (e.g., the intensity value for the intensity of the pixels of the lesion at the 10^{th} percentile), an LPB feature corresponding to a minimum value for a flat region; a distance to central foveal feature (which is a clinical feature), a texture feature in the form of lesion short run high gray level emphasis, a rim Feret max feature (which is a shape feature), a lesion Feret min (which is a shape feature), and an LPB feature corresponding to a “range” computed for an edge region. In other embodiments, some portion of these ten identified features may be used. For example, the first three, four, five, six, seven, eight, or nine of these ten features of this listing of features may be used as inputs for the customized ML model 134 in FIG. 1.

In some embodiments, the customized machine learning model may be, for example without limitation, a linear regression model with Elastic Net or an XGBoost algorithm.

Step 306 of process 300 includes predicting, via the customized machine learning model, a set of geographic atrophy (GA) progression parameters based on the input. In one or more embodiments, the set of GA progression parameters predicted by the customized machine learning model may include at least one of a baseline lesion area, a lesion growth rate, or a future lesion area.

Process 300, which may be implemented using prediction system 100 described with respect to FIGS. 1 and 2 provides an improvement to the technical field of predicting GA progression because accurate predictions may be made using the customized model, all while consuming reduced computing resources (e.g., processor, memory, etc.) and needing less time for the subset of features as compared to larger set of features. These improvements may in turn lead to technical improvements (e.g., identification of new image-based biomarkers) for GA evaluation, diagnosis, and treatment management.

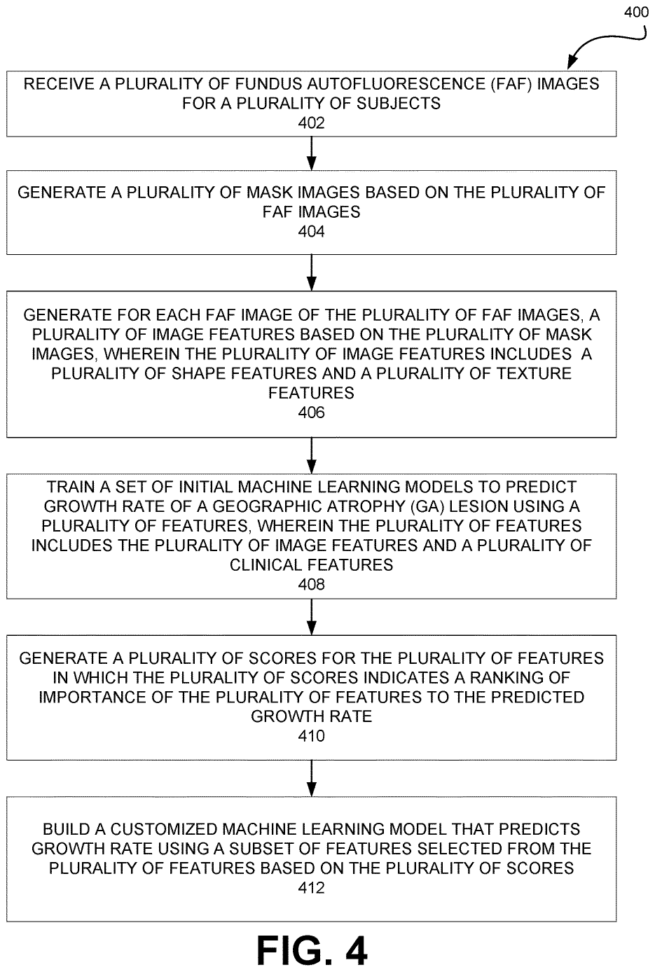

FIG. 4 is a flowchart of a process for generating a customized machine learning model in accordance with one or more example embodiments. Process 400 in FIG. 4 may be implemented using prediction system 100 described with respect to FIGS. 1 and 2. In one or more embodiments, at least some of the steps of the process 400 may be performed by the processors of a computer or a server implemented as part of prediction system 100. It is understood that additional steps may be performed before, during, or after the steps of process 400 discussed below. In addition, in some embodiments, one or more of the steps may also be omitted or performed in different orders.

Step 402 of process 400 includes receiving a plurality of fundus autofluorescence (FAF) images for a plurality of subjects.

Step 404 of process 400 includes generating a plurality of mask images based on the plurality of FAF images. In some embodiments, the plurality of mask images may include a lesion mask image, a rim mask image, and a background mask image.

Step 406 of process 400 includes generating, for each FAF image of the plurality of FAF images, a plurality of image features based on the plurality of mask images, wherein the plurality of image features includes a plurality of shape features and a plurality of texture features. In one or more embodiments, the plurality of shape features may include at least two or more of a measured GA lesion area, an identified number of lesions, a perimeter, a Feret max, a Feret min, and circularity. In some embodiments, the plurality of texture features may include radiomics features that include at least one of a set of gray-level co-occurrence matrix features, a set of gray level size zone matrix features, a set of gray level run length matrix features, a set of neighboring gray tone difference matrix features, or a set of gray level dependence matrix features. In one or more embodiments, the plurality of texture features may include a plurality of hyperautofluorescence (HA) features. In some embodiments, the plurality of image features may include a plurality of local binary pattern image features.

Step 408 of process 400 includes training a set of initial machine learning models to predict growth rate of a geographic atrophy (GA) lesion using a plurality of features, wherein the plurality of features includes the plurality of image features and a plurality of clinical features.

Step 410 of process 400 includes generating a plurality of scores for the plurality of features in which the plurality of scores indicates a ranking of importance of the plurality of features to the predicted growth rate.

Step 412 of process 400 includes building a customized machine learning model that predicts growth rate using a subset of features selected from the plurality of features based on the plurality of scores. The subset of features may include three, four, five, six, seven, eight, nine, ten, or some other number of features.

In one or more embodiments, the subset of features may include an LBP feature corresponding to a “range” computed for a corner region; an LPB feature corresponding to an inner quartile range (IQR) for a corner region; a low-luminance deficit feature (which is a clinical feature), an intensity feature for the lesion corresponding to the 10^{th} intensity percentile (e.g., the intensity value for the intensity of the pixels of the lesion at the 10^{th} percentile), an LPB feature corresponding to a minimum value for a flat region; a distance to central foveal feature (which is a clinical feature), a texture feature in the form of lesion short run high gray level emphasis, a rim Feret max feature (which is a shape feature), a lesion Feret min (which is a shape feature), and an LPB feature corresponding to a “range” computed for an edge region. In other embodiments, some portion of these ten identified features may be used. For example, the first three, four, five, six, seven, eight, or nine of these ten features of this listing of features may be used as inputs for the customized ML model 134 in FIG. 1.

Process 400, which may be implemented using prediction system 100 described with respect to FIGS. 1 and 2 provides an improvement to the technical field of predicting GA progression because accurate predictions may be made using the customized model, all while consuming reduced computing resources (e.g., processor, memory, etc.) and needing less time for the subset of features as compared to larger set of features. These improvements may in turn lead to technical improvements (e.g., identification of new image-based biomarkers) for GA evaluation, diagnosis, and treatment management.

### IV. Example Geographic Atrophy Progression Prediction

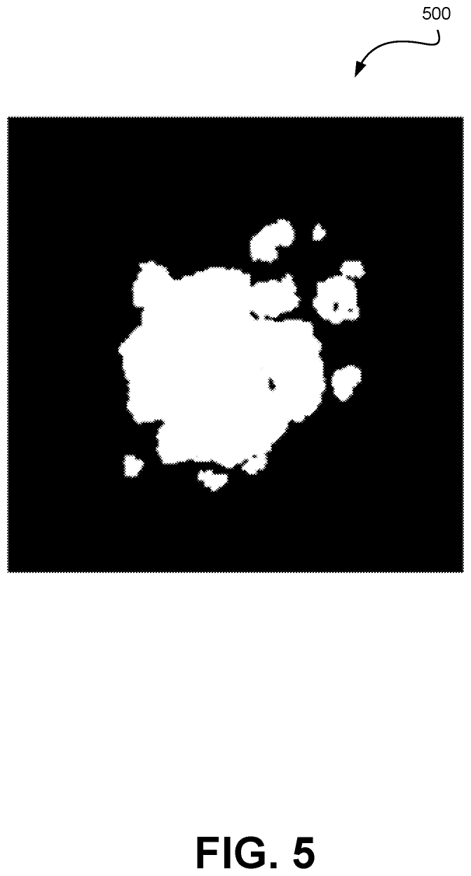

FIG. 5 is an example illustration of a lesion mask image in accordance with one or more embodiments. Lesion mask image 500 may be one example of a mask image of mask images 118 from FIG. 1.

In one example study, data from the study eyes of patients with bilateral GA enrolled in the lampalizumab phase 3 clinical trials (Chroma, NCT02247479; Spectri, NCT02247531) and in an accompanying observational study (Proxima A, NCT02479386) were used. Specifically, FAF images for these eyes were used. The inclusion criteria were bilateral GA, total GA area of 1 to 7 disc areas in the study eye, FAF pattern banded or diffuse in the study eye, and absence of past or current choroidal neovascularization in either eye. Only images acquired at the screening visit (e.g., baseline FAF images) were used in the prediction of GA growth and therefore unaffected by treatment status. Clinical features were identified from the data for this study. GA growth rate was defined as the annualized slope of a linear model (mm²/year).

Based on the baseline FAF images, various mask images were generated, including lesion mask images (e.g., lesion mask image 500 in FIG. 5), rim mask images (e.g., identifying a rim of about 500 micrometers around the GA lesion), and background mask images. These segmentations were used to generate (or derive) numerous image features (e.g., image features 122 from FIGS. 1 and 2). These image features were then used in combination with various clinical features as inputs into different models (e.g.,. various linear regression models—including linear regression with Lasso, linear regression with Ridge, linear regression with Elastic Net—and XGBoost). The performance of these models based on the different input combinations was evaluated and the contribution of the different features analyzed.

From the different available features, a subset of features was specifically selected to improve overall performance of a customized ML model (e.g., customized ML model 134 in FIG. 1), where accuracy is preserved while reducing the number of features and thereby processing resources that are needed to predict GA growth rate from FAF images.

Based on the study, in one or more embodiments, the subset of features may include an LBP feature corresponding to a “range” computed for a corner region; an LPB feature corresponding to an inner quartile range (IQR) for a corner region; a low-luminance deficit feature (which is a clinical feature), an intensity feature for the lesion corresponding to the 10^{th} intensity percentile (e.g., the intensity value for the intensity of the pixels of the lesion at the 10^{th} percentile), an LPB feature corresponding to a minimum value for a flat region; a distance to central foveal feature (which is a clinical feature), a texture feature in the form of lesion short run high gray level emphasis, a rim Feret max feature (which is a shape feature), a lesion Feret min (which is a shape feature), and an LPB feature corresponding to a “range” computed for an edge region. In other embodiments, some portion of these ten identified features may be used. For example, the first three, four, five, six, seven, eight, or nine of these ten features of this listing of features may be used as inputs for the customized ML model 134 in FIG. 1.

### V. Computer Implemented System

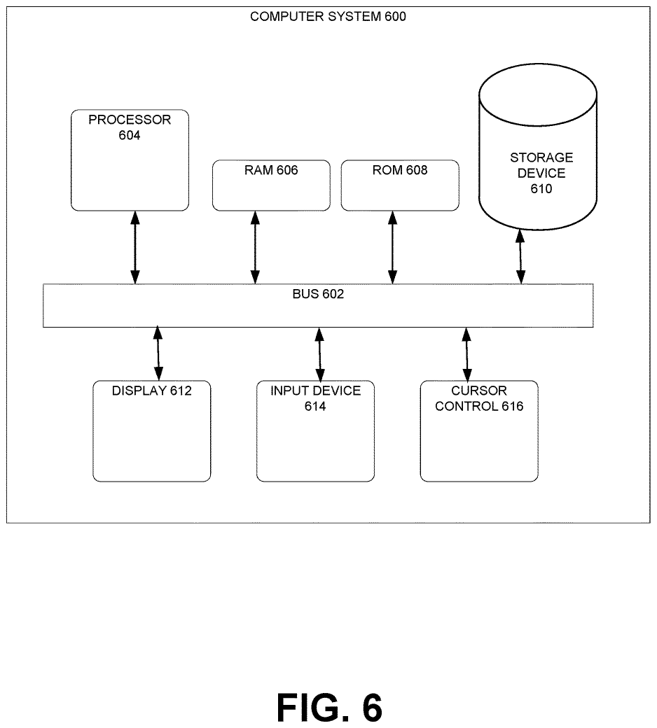

FIG. 6 is a block diagram of a computer system in accordance with various embodiments. Computer system 600 may be an example of one implementation for computing platform 102 described above in FIG. 1. In one or more examples, computer system 600 can include a bus 602 or other communication mechanism for communicating information, and a processor 604 coupled with bus 602 for processing information. In various embodiments, computer system 600 can also include a memory, which can be a random-access memory (RAM) 606 or other dynamic storage device, coupled to bus 602 for determining instructions to be executed by processor 604. Memory also can be used for storing temporary variables or other intermediate information during execution of instructions to be executed by processor 604. In various embodiments, computer system 600 can further include a read only memory (ROM) 608 or other static storage device coupled to bus 602 for storing static information and instructions for processor 604. A storage device 610, such as a magnetic disk or optical disk, can be provided and coupled to bus 602 for storing information and instructions.

In various embodiments, computer system 600 can be coupled via bus 602 to a display 612, such as a cathode ray tube (CRT) or liquid crystal display (LCD), for displaying information to a computer user. An input device 614, including alphanumeric and other keys, can be coupled to bus 602 for communicating information and command selections to processor 604. Another type of user input device is a cursor control 616, such as a mouse, a joystick, a trackball, a gesture input device, a gaze-based input device, or cursor direction keys for communicating direction information and command selections to processor 604 and for controlling cursor movement on display 612. This input device 614 typically has two degrees of freedom in two axes, a first axis (e.g., x) and a second axis (e.g., y), that allows the device to specify positions in a plane. However, it should be understood that input devices 614 allowing for three-dimensional (e.g., x, y and z) cursor movement are also contemplated herein.

Consistent with certain implementations of the present teachings, results can be provided by computer system 600 in response to processor 604 executing one or more sequences of one or more instructions contained in RAM 606. Such instructions can be read into RAM 606 from another computer-readable medium or computer-readable storage medium, such as storage device 610. Execution of the sequences of instructions contained in RAM 606 can cause processor 604 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 604 for execution. Such a medium can take many forms, including but not limited to, non-volatile media, volatile media, and transmission media. Examples of non-volatile media can include, but are not limited to, optical, solid state, magnetic disks, such as storage device 610. Examples of volatile media can include, but are not limited to, dynamic memory, such as RAM 606. Examples of transmission media can include, but are not limited to, coaxial cables, copper wire, and fiber optics, including the wires that comprise bus 602.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 604 of computer system 600 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 600 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 600, whereby processor 604 would execute the analyses and determinations provided by these engines, subject to instructions provided by any one of, or a combination of, the memory components RAM 606, ROM, 608, or storage device 610 and user input provided via input device 614.

VI. Example Context and Definitions

The disclosure is not limited to these example embodiments and applications or to the manner in which the example embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) may be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” may mean sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, may mean different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “lesion” may be a region in an organ or tissue that has suffered damage via injury or disease. This region may be a continuous or discontinuous region. For example, as used herein, a lesion may include multiple regions. A geographic atrophy (GA) lesion may be a region of the retina that has suffered chronic progressive degeneration. As used herein, a GA lesion may include one lesion (e.g., one continuous lesion region) or multiple lesions (e.g., discontinuous lesion region comprised of multiple, separate lesions).

As used herein, a “lesion area” may mean the total area covered by a lesion, whether that lesion be a continuous region or a discontinuous region.

As used herein, “longitudinal” may mean over a period of time. The period of time may be in days, weeks, months, years, or some other measure of time.

As used herein, a “growth rate” corresponding to a GA lesion may mean a longitudinal change in the lesion area of the GA lesion. This growth rate may also be referred to as a GA growth rate.

VII. Recitation of Embodiments

Embodiment 1: A method for evaluating geographic atrophy using a customized machine learning model, the method comprising: receiving a set of fundus autofluorescence (FAF) images for a subject; generating input for the machine learning model using the plurality of FAF images, wherein the input includes a subset of features that have been selected from a plurality of features, wherein the plurality of features includes a plurality of image features; wherein the plurality of image features includes at least one of a plurality of shape features and a plurality of texture features; wherein the subset of features includes fewer features than the plurality of features; wherein selection of the subset of features from the plurality of features is based on evaluating performance of a set of trained models that are used to process at least two different combinations of the plurality of features; and predicting, via the customized machine learning model, a set of geographic atrophy (GA) progression parameters based on the input.

Embodiment 2: The method of embodiment 1, wherein the set of GA progression parameters predicted by the customized machine learning model includes at least one of a baseline lesion area, a lesion growth rate, or a future lesion area.

Embodiment 3: The method of embodiment 1 or 2, wherein the plurality of shape features includes at least two or more of a measured GA lesion area, an identified number of lesions, a perimeter, a Feret max, a Feret min, and circularity.

Embodiment 4: The method of any one of embodiments 1-3, wherein generating the input comprises: generating a set of mask images based on the set of FAF images, wherein the set of mask images includes at least one of a lesion mask image, a rim mask image, a lesion and rim mask image, or a background mask image; and generating at least one feature of the subset of features using the set of mask images.

Embodiment 5: The method of embodiment 4, wherein generating the input further comprises: generating at least one shape feature that is included in the subset of features using the set of mask images.

Embodiment 6: The method of embodiment 4 or embodiment 5, wherein generating the input comprises: generating at least one hyperautofluorescence (HA) feature that is included in the subset of features using the set of mask images.

Embodiment 7: The method of any one of embodiments 4-6, wherein generating the input comprises: generating a set of radiomics features that is included in the subset of features using the set of mask images.

Embodiment 8: The method of any one of embodiments 4-7, wherein generating the input further comprises: computing a first local binary pattern for the lesion mask image and a second local binary pattern for the rim mask image; and generating a plurality of local binary pattern features that are included in the subset of features using the first local binary pattern and the second local binary pattern.

Embodiment 9: The method of embodiment 8, wherein the first local binary pattern is a uniform local binary pattern and wherein the second local binary pattern is a rotation-invariant local binary pattern.

Embodiment 10: The method of any one of embodiments 1-9, wherein the customized machine learning model is either a linear regression model with Elastic Net or an XGBoost algorithm.

Embodiment 11: The method of embodiment 1, wherein the plurality of texture features includes radiomics features that include at least one of a set of gray-level co-occurrence matrix features, a set of gray level size zone matrix features, a set of gray level run length matrix features, a set of neighboring gray tone difference matrix features, or a set of gray level dependence matrix features.

Embodiment 12: A method for generating a customized machine learning model, the method comprising: receiving a plurality of fundus autofluorescence (FAF) images for a plurality of subjects; generating a plurality of mask images based on the plurality of FAF images; generating, for each FAF image of the plurality of FAF images, a plurality of image features based on the plurality of mask images, wherein the plurality of image features includes a plurality of shape features and a plurality of texture features; training a set of initial machine learning models to predict growth rate of a geographic atrophy (GA) lesion using a plurality of features, wherein the plurality of features includes the plurality of image features and a plurality of clinical features; generating a plurality of scores for the plurality of features in which the plurality of scores indicates a ranking of importance of the plurality of features to the predicted growth rate; and building a customized machine learning model that predicts growth rate using a subset of features selected from the plurality of features based on the plurality of scores.

Embodiment 13: The method of embodiment 12, wherein the plurality of shape features includes at least two or more of a measured GA lesion area, an identified number of lesions, a perimeter, a Feret max, a Feret min, and circularity.

Embodiment 14: The method of embodiment 12 or embodiment 13, wherein the plurality of texture features includes radiomics features that include at least one of a set of gray-level co-occurrence matrix features, a set of gray level size zone matrix features, a set of gray level run length matrix features, a set of neighboring gray tone difference matrix features, or a set of gray level dependence matrix features.

Embodiment 15: The method of any one of embodiments 12-14, wherein the plurality of mask images includes a lesion mask image, a rim mask image, and a background mask image.

Embodiment 16: The method of any one of embodiments 12-15, wherein the plurality of texture features includes a plurality of hyperautofluorescence (HA) features.

Embodiment 17: The method of any one of embodiments 12-16, wherein the plurality of image features includes a plurality of local binary pattern image features.

Embodiment 18: A system for generating a customized machine learning model, the system comprising: at least one data processor; and at least one memory storing instructions, which when executed by the at least one data processor, cause the processor to: receive a plurality of fundus autofluorescence (FAF) images for a plurality of subjects; generate a plurality of mask images based on the plurality of FAF images; generate, for each FAF image of the plurality of FAF images, a plurality of image features based on the plurality of mask images, wherein the plurality of image features includes a plurality of shape features and a plurality of texture features; train a set of initial machine learning models to predict growth rate of a geographic atrophy (GA) lesion using a plurality of features, wherein the plurality of features includes the plurality of image features and a plurality of clinical features; generate a plurality of scores for the plurality of features in which the plurality of scores indicates a ranking of importance of the plurality of features to the predicted growth rate; and build a customized machine learning model that predicts growth rate using a subset of features selected from the plurality of features based on the plurality of scores.

Embodiment 19: The system of embodiment 18, wherein the plurality of shape features includes at least two or more of a measured GA lesion area, an identified number of lesions, a perimeter, a Feret max, a Feret min, and circularity.

Embodiment 20: The system of embodiment 18 or embodiment 19, wherein the plurality of texture features includes radiomics features that include at least one of a set of gray-level co-occurrence matrix features, a set of gray level size zone matrix features, a set of gray level run length matrix features, a set of neighboring gray tone difference matrix features, or a set of gray level dependence matrix features.

Embodiment 21: The system of any one of embodiments 18-20, wherein the plurality of mask images includes a lesion mask image, a rim mask image, and a background mask image.

Embodiment 22: The system of any one of embodiments 18-21, wherein the plurality of texture features includes a plurality of hyperautofluorescence (HA) features.

Embodiment 23: The system of any one of embodiments 18-22, wherein the plurality of image features includes a plurality of local binary pattern image features.

Embodiment 24: A system, including at least one data processor; and at least one memory storing instructions, which when executed by the at least one data processor, result in operations including the method of any of embodiments 1 to 17.

Embodiment 25: A non-transitory computer readable medium storing instructions, which when executed by at least one data processor, result in operations including the method of any of embodiments 1 to 17.

### VIII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

For example, the flowcharts and block diagrams described above illustrate the architecture, functionality, and/or operation of possible implementations of various method and system embodiments. Each block in the flowcharts or block diagrams may represent a module, a segment, a function, a portion of an operation or step, or a combination thereof. In some alternative implementations of an embodiment, the function or functions noted in the blocks may occur out of the order noted in the figures. For example, in some cases, two blocks shown in succession may be executed substantially concurrently. In other cases, the blocks may be performed in the reverse order. Further, in some cases, one or more blocks may be added to replace or supplement one or more other blocks in a flowchart or block diagram.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

What is claimed is:

1. A method for evaluating geographic atrophy using a customized machine learning model, the method comprising:
receiving a set of fundus autofluorescence (FAF) images for a subject;
generating input for the machine learning model using the plurality of FAF images,
wherein the input includes a subset of features that have been selected from a plurality of features,
wherein the plurality of features includes a plurality of image features;
wherein the plurality of image features includes at least one of a plurality of shape features and a plurality of texture features;
wherein the subset of features includes fewer features than the plurality of features;
wherein selection of the subset of features from the plurality of features is based on evaluating performance of a set of trained models that are used to process at least two different combinations of the plurality of features; and
predicting, via the customized machine learning model, a set of geographic atrophy (GA) progression parameters based on the input.

2. The method of claim 1, wherein the set of GA progression parameters predicted by the customized machine learning model includes at least one of a baseline lesion area, a lesion growth rate, or a future lesion area.

3. The method of claim 1, wherein the plurality of shape features includes at least two or more of a measured GA lesion area, an identified number of lesions, a perimeter, a Feret max, a Feret min, and circularity.

4. The method of claim 1, wherein generating the input comprises:
generating a set of mask images based on the set of FAF images, wherein the set of mask images includes at least one of a lesion mask image, a rim mask image, a lesion and rim mask image, or a background mask image; and
generating at least one feature of the subset of features using the set of mask images.

5. The method of claim 4, wherein generating the input further comprises:
generating at least one shape feature that is included in the subset of features using the set of mask images.

6. The method of claim 4, wherein generating the input comprises:
generating at least one hyperautofluorescence (HA) feature that is included in the subset of features using the set of mask images.

7. The method of claim 4, wherein generating the input comprises:
generating a set of radiomics features that is included in the subset of features using the set of mask images.

8. The method of claim 4, wherein generating the input further comprises:
computing a first local binary pattern for the lesion mask image and a second local binary pattern for the rim mask image; and
generating a plurality of local binary pattern features that are included in the subset of features using the first local binary pattern and the second local binary pattern.

9. The method of claim 8, wherein the first local binary pattern is a uniform local binary pattern and wherein the second local binary pattern is a rotation-invariant local binary pattern.

10. The method of claim 1, wherein the customized machine learning model is either a linear regression model with Elastic Net or an XGBoost algorithm.

11. The method of claim 1, wherein the plurality of texture features includes radiomics features that include at least one of a set of gray-level co-occurrence matrix features, a set of gray level size zone matrix features, a set of gray level run length matrix features, a set of neighboring gray tone difference matrix features, or a set of gray level dependence matrix features.

12. A method for generating a customized machine learning model, the method comprising:
receiving a plurality of fundus autofluorescence (FAF) images for a plurality of subjects;
generating a plurality of mask images based on the plurality of FAF images;
generating, for each FAF image of the plurality of FAF images, a plurality of image features based on the plurality of mask images, wherein the plurality of image features includes a plurality of shape features and a plurality of texture features;
training a set of initial machine learning models to predict growth rate of a geographic atrophy (GA) lesion using a plurality of features, wherein the plurality of features includes the plurality of image features and a plurality of clinical features;
generating a plurality of scores for the plurality of features in which the plurality of scores indicates a ranking of importance of the plurality of features to the predicted growth rate; and
building a customized machine learning model that predicts growth rate using a subset of features selected from the plurality of features based on the plurality of scores.

13. The method of claim 12, wherein the plurality of shape features includes at least two or more of a measured GA lesion area, an identified number of lesions, a perimeter, a Feret max, a Feret min, and circularity.

14. The method of claim 12, wherein the plurality of texture features includes radiomics features that include at least one of a set of gray-level co-occurrence matrix features, a set of gray level size zone matrix features, a set of gray level run length matrix features, a set of neighboring gray tone difference matrix features, or a set of gray level dependence matrix features.

15. The method of claim 12, wherein the plurality of mask images includes a lesion mask image, a rim mask image, and a background mask image.

16. The method of claim 12, wherein the plurality of texture features includes a plurality of hyperautofluorescence (HA) features.

17. The method of claim 12, wherein the plurality of image features includes a plurality of local binary pattern image features.

18. A system for generating a customized machine learning model, the system comprising:
at least one data processor; and at least one memory storing instructions, which when executed by the at least one data processor, cause the processor to:
receive a plurality of fundus autofluorescence (FAF) images for a plurality of subjects;
generate a plurality of mask images based on the plurality of FAF images;
generate, for each FAF image of the plurality of FAF images, a plurality of image features based on the plurality of mask images, wherein the plurality of image features includes a plurality of shape features and a plurality of texture features;
train a set of initial machine learning models to predict growth rate of a geographic atrophy (GA) lesion using a plurality of features, wherein the plurality of features includes the plurality of image features and a plurality of clinical features;
generate a plurality of scores for the plurality of features in which the plurality of scores indicates a ranking of importance of the plurality of features to the predicted growth rate; and
build a customized machine learning model that predicts growth rate using a subset of features selected from the plurality of features based on the plurality of scores.

19. The system of claim 18, wherein the plurality of shape features includes at least two or more of a measured GA lesion area, an identified number of lesions, a perimeter, a Feret max, a Feret min, and circularity.

20. The system of claim 18, wherein the plurality of texture features includes radiomics features that include at least one of a set of gray-level co-occurrence matrix features, a set of gray level size zone matrix features, a set of gray level run length matrix features, a set of neighboring gray tone difference matrix features, or a set of gray level dependence matrix features.
